(19) United States
(12) Patent Application Publication
(10) Pub. No.: US 2008/0194623 A1
(43) Pub. Date: Aug. 14, 2008

(54) Method for the Synthesis of Quinoline Derivatives

(76) Inventors: Clifford S. Labaw, Philadelphia, PA (US); Peng Liu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/997,378

(22) PCT Filed: Aug. 2, 2006
(86) PCT No.: PCT/US06/30042

§ 371 Date: Jan. 30, 2008

Related U.S. Application Data
(60) Provisional application No. 60/704,540, filed on Aug. 2, 2005.

(51) Int. Cl. A61K 31/47; C07D 215/52

(52) U.S. Cl. 514/312; 546/156

(57) ABSTRACT

This invention relates to novel intermediates and processes for preparing pharmaceutically active quinoline compounds, including (−)-(S)—N-(α-ethylbenzyl)-3-hydroxy-2-phenylquinoline-4-carboxamide.

### FIELD OF THE INVENTION

This invention relates to novel processes and intermediates useful for preparing pharmaceutically active quinoline compounds, including (−)-(S)—N-(α-ethylbenzyl)-3-hydroxy-2-phenylquinoline-4-carboxamide.

### BACKGROUND OF THE INVENTION

This invention describes methods for the preparation of compounds of the structural formula (I)

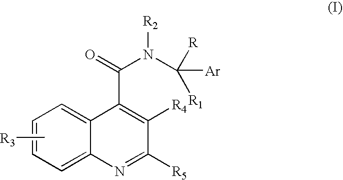

or a pharmaceutically acceptable salt form thereof, wherein:

Ar is an optionally substituted phenyl group, or a naphthyl or C₅₋₇ cycloalkdienyl group, or an optionally substituted single or fused ring heterocyclic group, having aromatic character, containing from 5 to 12 ring atoms and comprising up to four hetero-atoms selected from S, O, N;

R is linear or branched C₁₋₈ alkyl, C₃₋₇ cycloalkyl, C₄₋₇ cycloalkylalkyl, an optionally substituted phenyl group or a phenyl C₁₋₆ alkyl group, an optionally substituted five-membered heteroaromatic ring comprising up to four heteroatom selected from O and N, hydroxy C₁₋₆ alkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl; or is a group —(CH₂)ₚ— when cyclized onto Ar, where p is 2 or 3;

R₁ and R₂, which may be the same or different, are independently hydrogen or C₁₋₆ linear or branched alkyl, or together form a —(CH₂)ₙ— group in which n represents 3, 4, or 5; or R₁ together with R forms a group —(CH₂)_{q}—, in which q is 2, 3, 4 or 5;

R₃ may be hydrogen, C₁₋₆ linear or branched alkyl, C₁₋₆ alkenyl, aryl, halogen, nitro, cyano, carboxy, carboxamido, sulphonamido, trifluoromethyl, amino, mono- and di-C₁₋₆ alkylamino, —O(CH₂)ᵣ—NT₂, in which r is 2, 3, or 4 and T is C₁₋₆ alkyl or it forms a heterocyclic group

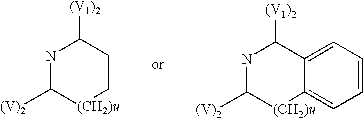

in which V and V₁ are hydrogen and u is 0, 1 or 2;

R₄ is hydroxyl;

R₅ is branched or linear C₁₋₆ alkyl, C₃₋₇ cycloalkyl, C₄₋₇ cycloalkylalkyl, optionally substituted aryl, wherein the optional substituent is one of hydroxy, halogen, C₁₋₆ alkoxy or C₁₋₆ alkyl, or an optionally substituted single or fused ring heterocyclic group, having aromatic character, containing from 5 to 12 ring atoms and comprising up to four hetero-atoms in the or each ring selected from S, O, N. Said compounds are NK-3 antagonists and are useful in treating pulmonary disorders (asthma, chronic obstructive pulmonary diseases (COPD), airway hyperreactivity, cough), skin disorders and itch (for example, atopic dermatitis and cutaneous wheal and flare), neurogenic inflammation, CNS disorders (Parkinson's disease, movement disorders, anxiety), convulsive disorders (for example epilepsy), renal disorders, urinary incontinence, ocular inflammation, inflammatory pain, eating disorders (food intake inhibition), allergic rhinitis, neurodegenerative disorders (for example Alzheimer's disease), psoriasis, Huntington's disease, and depression. A particularly useful NK-3 receptor antagonist falling within the genus of formula (I) is (−)-(S)—N-(α-ethylbenzyl)-3-hydroxy-2-phenylquinoline-4-carboxamide. Such compounds, and methods for preparing the compounds, are disclosed in WO 95/32948, WO96/02509 and U.S. Pat. No. 6,335,448, the disclosures of which are incorporated herein by reference.

Previous published routes for the synthesis of compounds of formula (I), such as (−)-(S)—N-(α-ethylbenzyl)-3-hydroxy-2-phenylquinoline-4-carboxamide, have certain difficulties associated with them which might limit their appeal, especially where scaled-up procedures are desired. For example, a method previously disclosed in WO 95/32948 for the production of (−)-(S)—N-(α-ethylbenzyl)-3-hydroxy-2-phenylquinoline-4-carboxamide described the coupling of a carboxylic acid 1 with the α-benzylamine 2 in the presence of DCC to give (−)-(S)—N-(α-ethylbenzyl)-3-hydroxy-2-phenylquinoline-4-carboxamide 3 which was reported to also generate the side product 4 (as reported in U.S. Pat. No. 6,335,448). (See Scheme 1).

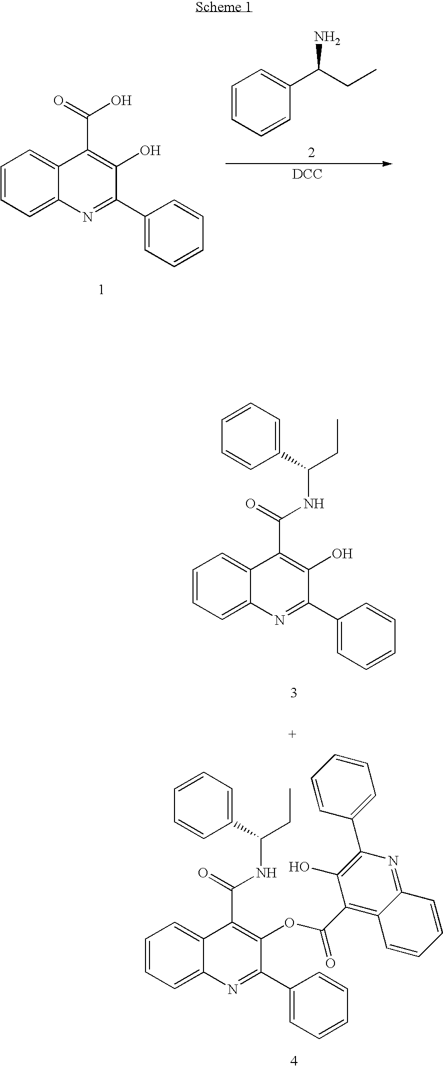

Another procedure disclosed for the production of a compound of formula (I), and specifically exemplified for (−)-(S)—N-(α-ethylbenzyl)-3-hydroxy-2-phenylquinoline-4-carboxamide was described more recently in U.S. Pat. No. 6,335,448 and is shown in Scheme 2 and described here. In the procedure described in U.S. Pat. No. 6,335,448, the production of (−)-(S)—N-(α-ethylbenzyl)-3-hydroxy-2-phenylquinoline-4-carboxamide 3 was reported to occur when the 3-hydroxy-4-quinoline carboxylic acid 1 was reacted with triethylamine and then SOCl₂, followed by treatment with the α-benzylamine 2 to render (−)-(S)—N-(α-ethylbenzyl)-3-hydroxy-2-phenylquinoline-4-carboxamide as its free base in a solution yield of 80%, which was subsequently converted to its HCl salt and isolated in 72% yield. One of the problems associated with this procedure is the putative coproduction of an unreactive cyclotrimer impurity 5.

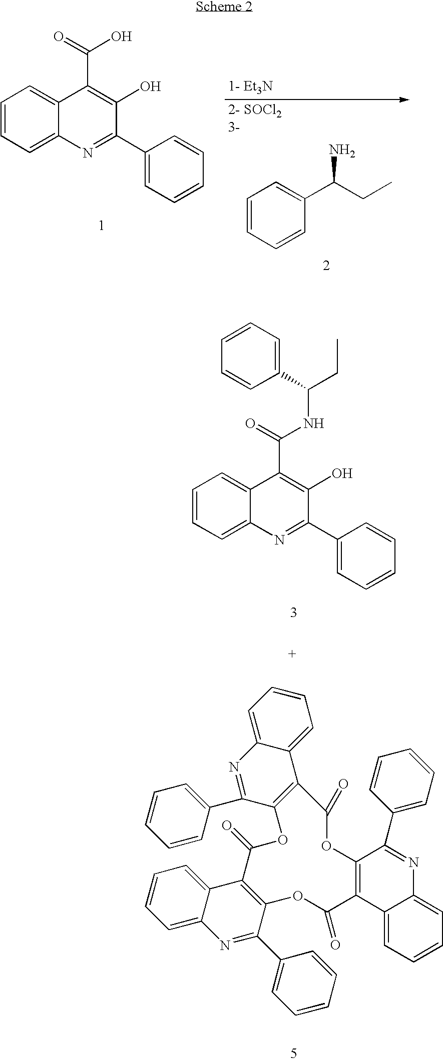

Given the known syntheses for quinoline NK-3 receptor antagonists of formula (I), there is still an unmet need for a synthetic procedure which can be efficiently scaled up in high yield with suppression of unwanted, difficult to remove side products, such as the unreactive cyclotrimer mentioned, supra. The present invention provides new synthetic processes for the preparation of (−)-(S)—N-(α-ethylbenzyl)-3-hydroxy-2-phenylquinoline-4-carboxamide and related compounds, which suppresses undesired and unreactive cyclotrimers, thus resulting in a straightforward product purification with high product yield, high purity, and shortened production times.

### SUMMARY OF THE INVENTION

The object of this invention is to provide novel processes for the production of compounds of formula (I), as well as novel intermediates useful in the preparation of compounds of formula (I). Accordingly, in one aspect, this invention describes a method of preparation of a compound of formula (I)

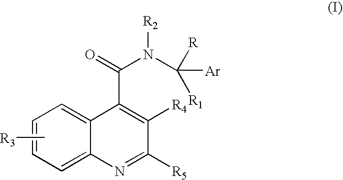

or a pharmaceutically acceptable salt form thereof, wherein:

Ar is an optionally substituted phenyl group, or a naphthyl or C₅₋₇ cycloalkdienyl group, or an optionally substituted single or fused ring heterocyclic group, having aromatic character, containing from 5 to 12 ring atoms and comprising up to four hetero-atoms selected from S, O, N;

R is linear or branched C₁₋₈ alkyl, C₃₋₇ cycloalkyl, C₄₋₇ cycloalkylalkyl, an optionally substituted phenyl group or a phenyl C₁₋₆ alkyl group, an optionally substituted five-membered heteroaromatic ring comprising up to four heteroatom selected from O and N, hydroxy C₁₋₆ alkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl; or is a group —(CH₂)ₚ— when cyclized onto Ar, where p is 2 or 3;

R₁ and R₂, which may be the same or different, are independently hydrogen or C₁₋₆ linear or branched alkyl, or together form a —(CH₂)ₙ— group in which n represents 3, 4, or 5; or R₁ together with R forms a group —(CH₂)_{q}—, in which q is 2, 3, 4 or 5;

R₃ may be hydrogen, C₁₋₆ linear or branched alkyl, C₁₋₆ alkenyl, aryl, halogen, nitro, cyano, carboxy, carboxamido, sulphonamido, trifluoromethyl, amino, mono- and di-C₁₋₆ alkylamino, —O(CH₂)ᵣ—NT₂, in which r is 2, 3, or 4 and T is C₁₋₆ alkyl or it forms a heterocyclic group

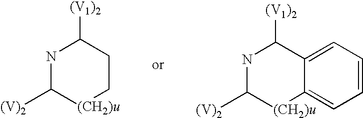

in which V and V₁ are hydrogen and u is 0, 1 or 2;

R₄ is hydroxyl;

R₅ is branched or linear C₁₋₆ alkyl, C₃₋₇ cycloalkyl, C₄₋₇ cycloalkylalkyl, optionally substituted aryl, wherein the optional substituent is one of hydroxy, halogen, C₁₋₆ alkoxy or C₁₋₆ alkyl, or an optionally substituted single or fused ring heterocyclic group, having aromatic character, containing from 5 to 12 ring atoms and comprising up to four hetero-atoms selected from S, O, N, comprising:

a) contacting a compound of formula (IV) with 1,1′-carbonyldiimidazole

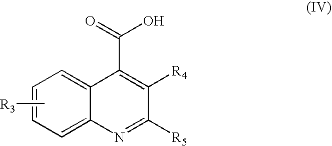

in the presence of optional base and optional solvent; and
b) contacting the product of step a) with a compound of formula (III)

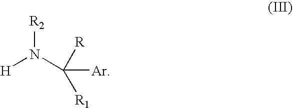

In some embodiments, the compound of formula (I) is (−)-(S)—N-(α-ethylbenzyl)-3-hydroxy-2-phenylquinoline-4-carboxamide or a salt or solvate thereof, the compound of formula (IV) is 3-hydroxy-2-phenyl-4-quinolinecarboxylic acid or a salt or solvate thereof, and the compound of formula (III) is S-1-phenylpropylamine or a salt or solvate thereof.

In some embodiments, a solvent is used in step a) of the methods of this invention and in some embodiments said solvent comprises an ether, aromatic hydrocarbon, alkylnitrile, or ester. In certain embodiments, said solvent comprises tetrahydrofuran, acetonitrile, toluene, or n-propylacetate. In some embodiments, the solvent is acetonitrile.

In certain aspects of this invention, step a) is performed in the presence of a base. In some embodiments, the base is an amine. In some embodiments, the amine is selected from the group consisting of 2,6-lutidine, 2,4,6-collidine, 2,3-lutidine, pyrazine, pyridine, N-methylpyrrole, DBN (1,5-Diazabicyclo[4.3.0]non-5-ene), DBU (diazabicyclo[5.4.0]undec-7-ene), imidazole, DMAP (4-dimethylaminopyridine), triethylamine, N,N-dimethylethylamine, N-methylmorpholine, diisopropylethylamine, diisopropylamine, 2,6-dimethylpiperidine, tributylamine, and dicyclohexylamine, and combinations thereof. In some embodiments, the amine is selected from the group consisting of imidazole, N-methylmorpholine, or triethylamine, or a combination thereof. In some embodiments, the amine is triethylamine. In some embodiments, the triethylamine is present in greater than 1 equivalent.

In some embodiments, an acid is added to the reaction mixture of step b). In some embodiments the acid is glacial acetic acid.

In some embodiments, the method of this invention uses a solvent in step a), wherein said solvent comprises acetonitrile and the reaction mixture of step a) further comprises 3-hydroxy-2-phenyl-4-quinolinecarboxylic acid, 1,1′-carbonyldiimidazole and triethylamine and is heated to about 40 to 50° C.; and wherein said triethylamine is present in more than 1 equivalent. In some embodiments for step b) of the methods of this invention, S-1-phenylpropylamine is added to the reaction mixture from step a) and heated to about 70 to 75° C. In some embodiments, the reaction mixture from step b) is treated with glacial acetic acid.

In certain embodiments, this invention describes a compound of formula (II)

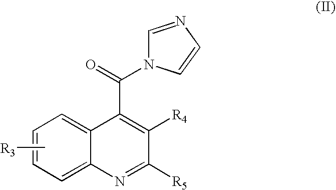

wherein R₃, R₄ and R₅ are as defined in claim 1.

In some embodiments, the compound of formula (II) of claim is 4-(1H-imidazol-1-ylcarbonyl)-2-phenyl-3-quinolinol.

In some aspects, this invention describes a mixture containing 4-(1H-imidazol-1-ylcarbonyl)-2-phenyl-3-quinolinol and 3-hydroxy-2-phenyl-4-quinolinecarboxylic acid, wherein the molar ratio of 4-(1H-imidazol-1-ylcarbonyl)-2-phenyl-3-quinolinol and 3-hydroxy-2-phenyl-4-quinolinecarboxylic acid is greater than ½.

In certain embodiments, this invention describes a method of preparing a compound of formula (I), wherein said method comprises contacting a compound of formula (IV)

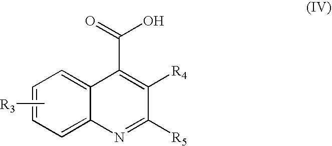

with 1,1′ carbonyldiimidazole.

In some embodiments, the compound of formula (IV) is 3-hydroxy-2-phenyl-4-quinolinecarboxylic acid.

In some aspects, this invention describes a method of preparing a compound of formula (I),

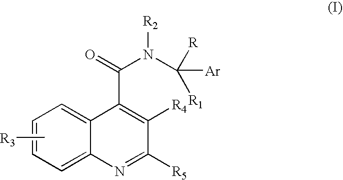

comprising the contacting of a compound of formula (II)

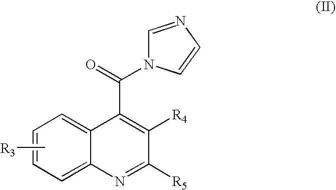

with a compound of formula (III) of claim 1,

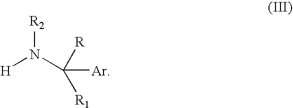

wherein Ar is an optionally substituted phenyl group, or a naphthyl or C₅₋₇ cycloalkdienyl group, or an optionally substituted single or fused ring heterocyclic group, having aromatic character, containing from 5 to 12 ring atoms and comprising up to four hetero-atoms selected from S, O, N;

R is linear or branched C₁₋₈ alkyl, C₃₋₇ cycloalkyl, C₄₋₇ cycloalkylalkyl, an optionally substituted phenyl group or a phenyl C₁₋₆ alkyl group, an optionally substituted five-membered heteroaromatic ring comprising up to four heteroatom selected from O and N, hydroxy C₁₋₆ alkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl; or is a group —(CH₂)ₚ— when cyclized onto Ar, where p is 2 or 3;

R₁ and R₂, which may be the same or different, are independently hydrogen or C₁₋₆ linear or branched alkyl, or together form a —(CH₂)ₙ— group in which n represents 3, 4, or 5; or R₁ together with R forms a group —(CH₂)_{q}—, in which q is 2, 3, 4 or 5;

R₃ may be hydrogen, C₁₋₆ linear or branched alkyl, C₁₋₆ alkenyl, aryl, halogen, nitro, cyano, carboxy, carboxamido, sulphonamido, trifluoromethyl, amino, mono- and di-C₁₋₆ alkylamino, —O(CH₂)ᵣ—NT₂, in which r is 2, 3, or 4 and T is C₁₋₆ alkyl or it forms a heterocyclic group

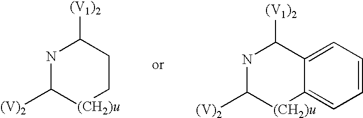

in which V and V₁ are hydrogen and u is 0, 1 or 2;

R₄ is hydroxyl;

R₅ is branched or linear C₁₋₆ alkyl, C₃₋₇ cycloalkyl, C₄₋₇ cycloalkylalkyl, optionally substituted aryl, wherein the optional substituent is one of hydroxy, halogen, C₁₋₆ alkoxy or C₁₋₆ alkyl, or an optionally substituted single or fused ring heterocyclic group, having aromatic character, containing from 5 to 12 ring atoms and comprising up to four hetero-atoms selected from S, O, N.

In certain aspects, this invention describes a method of purification of (−)-(S)—N-(α-ethylbenzyl)-3-hydroxy-2-phenylquinoline-4-carboxamide comprising heating a mixture of (−)-(S)—N-(α-ethylbenzyl)-3-hydroxy-2-phenylquinoline-4-carboxamide in a solvent system comprising an ester followed by cooling and isolating the product.

In some embodiments, said ester is n-propyl acetate.

In some embodiments, the purification mixture further comprises activated carbon.

### DETAILED DESCRIPTION OF THE INVENTION

While processes for the production of compounds of formula (I) are known, in particular as described for a compound of formula (I) which is (−)-(S)—N-(α-ethylbenzyl)-3-hydroxy-2-phenylquinoline-4-carboxamide; the discovery of alternative procedures is highly desirable, especially where the previous procedures resulted in compromised yields and/or hard to separate by-products. In particular, it appears that at least one of the previously reported procedures for the production of compounds of formula (I) and, in particular, for (−)-(S)—N-(α-ethylbenzyl)-3-hydroxy-2-phenylquinoline-4-carboxamide, the product was derived, at least in part, via cyclooligomerized intermediates which could then proceed to completed products (see U.S. Pat. No. 6,335,448). However, at least one cyclooligomer product, the cyclotrimer of a compound of formula (I) appears resistant to further reaction and thus can result in a decrease of yield for the reaction procedure. In U.S. Pat. No. 6,335,448, the difficulty of coupling α-hydroxy acid was noted, and that reference taught the formation of the reactive cyclooliogmeric intermediates as a way to obviate the difficult reactivity of those systems. The current invention describes a method to prepare the desired compounds of formula (I) via a coupling step, while both avoiding the reported low yields and hard to purify side products (e.g. DCU) of conventional methods (e.g. DCC coupling) while at the same time suppressing the cyclooligomerization reaction which can apparently form at least one relatively non-reactive species. Apparently, in contrast to previous disclosed methods which rely upon cyclooligomerization as reactive intermediates formed upon the way to the final product (as described in U.S. Pat. No. 6,335,448), the presently disclosed method largely or completely suppresses such formation, and thus minimizes or avoids the formation of the unreactive, cyclotrimerized intermediate, but still delivers a very high yield of product with the added advantage of improved ease of separation. Accordingly, the present invention provides processes for the production of a compound of formula

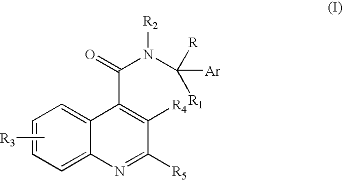

or a pharmaceutically acceptable salt form thereof, wherein:

Ar is an optionally substituted phenyl group, or a naphthyl or C₅₋₇ cycloalkdienyl group, or an optionally substituted single or fused ring heterocyclic group, having aromatic character, containing from 5 to 12 ring atoms and comprising up to four hetero-atoms selected from S, O, N;

R is linear or branched C₁₋₈ alkyl, C₃₋₇ cycloalkyl, C₄₋₇ cycloalkylalkyl, an optionally substituted phenyl group or a phenyl C₁₋₆ alkyl group, an optionally substituted five-membered heteroaromatic ring comprising up to four heteroatom selected from O and N, hydroxy C₁₋₆ alkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl; or is a group —(CH₂)ₚ— when cyclized onto Ar, where p is 2 or 3;

R₁ and R₂, which may be the same or different, are independently hydrogen or C₁₋₆ linear or branched alkyl, or together form a —(CH₂)ₙ— group in which n represents 3, 4, or 5; or R₁ together with R forms a group —(CH₂)_{q}—, in which q is 2, 3, 4 or 5;

R₃ may be hydrogen, C₁₋₆ linear or branched alkyl, C₁₋₆ alkenyl, aryl, halogen, nitro, cyano, carboxy, carboxamido, sulphonamido, trifluoromethyl, amino, mono- and di-C₁₋₆ alkylamino, —O(CH₂)ᵣ—NT₂, in which r is 2, 3, or 4 and T is C₁₋₆ alkyl or it forms a heterocyclic group

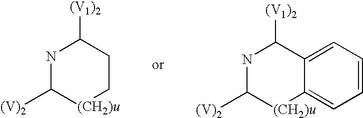

in which V and V₁ are hydrogen and u is 0, 1 or 2;

R₄ is hydroxyl;

R₅ is branched or linear C₁₋₆ alkyl, C₃₋₇ cycloalkyl, C₄₋₇ cycloalkylalkyl, optionally substituted aryl, wherein the optional substituent is one of hydroxy, halogen, C₁₋₆ alkoxy or C₁₋₆ alkyl, or an optionally substituted single or fused ring heterocyclic group, having aromatic character, containing from 5 to 12 ring atoms and comprising up to four hetero-atoms selected from S, O, N

or a pharmaceutically acceptable salt or solvate thereof, comprising:

a) contacting a compound of formula (II)

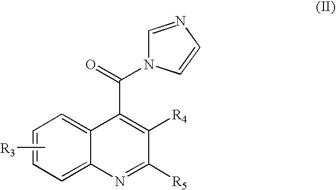

b) with a compound of formula (III)

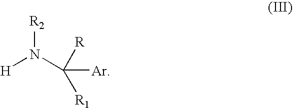

In a further embodiment of this invention, a compound of formula (II) is prepared via contacting a compound of formula (IV) with 1,1′-carbonyldiimidazole

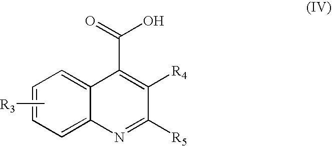

in the presence of optional base and optional solvent.
In some embodiments, this invention describes the preparation of a compound of formula (I), comprising:
a) the contacting a compound of formula (IV) with 1,1′-carbonyldiimidazole

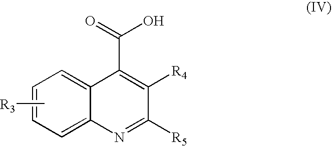

in the presence of optional base and optional solvent; and
b) contacting the product of step a) with a compound of formula (III)

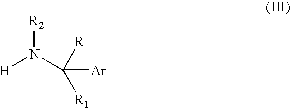

In some embodiments, the processes of this invention are applied for the production of a compound of formula (I), wherein Ar is phenyl, optionally substituted by C₁₋₆ alkyl or halogen; thienyl or a C₅₋₇ cycloalkdienyl group;

R is C₁₋₆ alkyl, C₁₋₆ alkoxycarbonyl, C₁₋₆ alkylcarbonyl, hydroxy C₁₋₆ alkyl;
R₁ and R₂ are each hydrogen or C₁₋₆ alkyl; R₃ is hydrogen, hydroxy, halogen, C₁₋₆ alkoxy, C₁₋₆ alkyl; R₄ is hydrogen, C₁₋₆ alkyl, C₁₋₆ alkoxy, hydroxy, amino, halogen, aminoalkoxy, mono- or di-alkylaminoalkoxy, mono- or di-alkylaminoalkyl, phthaloylalkoxy, mono- or di-alkylaminoacylamino and acylamino; and R₅ is phenyl, thienyl, furyl, pyrroyl and thiazolyl.

In some embodiments, the processes of this invention are applied to the production of a compound of formula (I), wherein Ar is phenyl; R is ethyl; R₁ and R₂ are each hydrogen; R₃ is hydrogen; and R₅ is phenyl.

In certain embodiments, this invention describes processes for the preparation of (−)-(S)—N-(α-ethylbenzyl)-3-hydroxy-2-phenylquinoline-4-carboxamide.

In some embodiments, this invention describes a compound of formula (II). In some embodiments, the compound of formula (II) is 4-(1H-imidazol-1-ylcarbonyl)-2-phenyl-3-quinolinol.

In some embodiments of this invention, the compound of formula (II) is not isolated.

In some aspects, this invention describes a process for the production of a compound of formula (I), or any of its structural embodiments described herein, wherein for step a), a base is used. In some embodiments, the base is organic. In some embodiments, the organic base is nitrogenous. In certain aspects, the nitrogeneous base contains at least one secondary or tertiary amine. In particular embodiments, where the amine is a secondary amine, the amine may be a dialkylamine, wherein said alkyl groups are each independently C₁₋₆ alkyl, wherein the C₁₋₆ alkyl groups maybe branched, straight, or together form a ring containing a total of from 3 to 7 atoms (wherein said ring maybe further substituted with from 1 to 3 C₁₋₃ alkyl groups). In some embodiments, the secondary amine may be part of a five-member heteroaromatic ring (wherein said ring maybe optionally substituted with up to 2 substitutents selected from C₁₋₃alkyl or halogen). In embodiments wherein the amine used is a tertiary amine, the amine maybe a trialkylamine, wherein each alkyl group is independently selected from C₁₋₆alkyl wherein the C₁₋₆ alkyl groups may be branched, straight, or two together form a ring containing a total of from 3 to 7 atoms (wherein said ring maybe further substituted with from 1- to 3-C₁₋₃ alkyl groups). In other embodiments, the tertiary amine maybe a backbone atom in a 5 or 6 member heteroaromatic, wherein said heteroaromatic is optionally substituted with up to 3 groups selected from C₁₋₃alkyl, dimethylamino, or halogen. Some non-limiting examples of some amines useful for this invention are 2,6-lutidine, 2,4,6-collidine, 2,3-lutidine, pyrazine, pyridine, N-methylpyrrole, DBN (1,5-Diazabicyclo[4.3.0]non-5-ene), DBU (diazabicyclo[5.4.0]undec-7-ene), imidazole, DMAP (4-dimethylaminopyridine), triethylamine, N,N-dimethylethylamine, N-methylmorpholine, diisopropylethylamine, diisopropylamine, 2,6-dimethylpiperidine, tributylamine, dicyclohexylamine, and the like, or combinations thereof. In some embodiments, the amine is imidazole, N-methylmorpholine, or triethylamine. In some embodiments, triethylamine is used and is present in greater than 1 equivalent. In some embodiments, the triethylamine is present in from 1.2 to 1.4 equivalents. In some embodiments, triethylamine is used and is present in about 1.3 equivalents.

In certain embodiments, the contacting of the compound of formula (IV) or any of its structural embodiments described herein, with 1,1′-carbonyldiimidazole and a nitrogeneous base takes place in a solvent. In some embodiments, the solvent used is an organic, aprotic solvent. In some embodiments, the solvent used is an ether, aromatic hydrocarbon, alkylnitrile, or ester. In certain embodiments, the solvent comprises tetrahydrofuran, acetonitrile, toluene, or n-propylacetate.

In some embodiments, the solvent used is acetonitrile.

In some embodiments, the ratio of solvent to reactant is optimized with a general preference noted where solvent to reactant ratios are kept to a minimum. For example, the higher the ratio of solvent used, the more waste effluent that is generated, and additionally, less effective product recovery or lower yield can sometimes be observed. Accordingly, in certain embodiments, the amount of solvent to reactant of formula (IV) on a weight per weight basis, is kept below 10:1. In certain embodiments, the ratio is kept below 7:1; or 6:1; or 5:1 or 4:1; or less than 4:1.

In one aspect of certain processes of this invention, the compound of formula (IV) or any of its structural embodiments described herein, is put in a solvent, then treated with the nitrogeneous base and 1,1′-carbonyldiimidazole. In some embodiments, the mixture containing the compound of formula (IV), the nitrogeneous base and 1,1′-carbonyldiimidazole are heated above 25° C. In some embodiments, the mixture is heated above 30° C., or above 40° C. or between about 40° C. to about 50° C. In some embodiments, the mixture containing the compound of formula (IV), the nitrogeneous base and 1,1′-carbonyldiimidazole is kept under an inert atmosphere. In some embodiments, the inert atmosphere is nitrogen.

In some aspects, the reaction to form the acylimidazole intermediate of formula (II) is monitored for a desired level of completion. In some embodiments, the desired level of completion is at least 50%, or at least 60%, or at least 70%, or at least 80%, or at least 90%, or at least 95%, or at least 97%, or at least 98%, or at least 99%. In some embodiments, the reaction is monitored for completion by removing a sample of the reaction mixture and quenching with an organic alcohol or amine, and subjecting the quenched sample to an analytical procedure whereby the ratio of the starting material to the corresponding ester or amide is determined.

In certain embodiments, the compound of formula (II) is added to the amine (III). In some embodiments, the amine (III) is added to the compound of formula (II). In some embodiments, the amine (III) is added neat or essentially neat to the mixture containing the compound of formula (II). In some embodiments, the compound of formula (II) is added as a mixture or solution. In some embodiments, the amine of formula (III) is added to the reaction mixture containing (II), which reaction mixture is above 20° C., or above 30° C., or above 40° C., or about 40 to 50° C.

In some embodiments, the mixture or solution containing (II) and (III) is heated at a temperature above 30° C., or above 40° C., or above 50° C., or above 60° C., or above 70° C., or above 75° C. or about from 70 to 75° C. In some embodiments, the reaction is monitored for substantial completion and diluted with an acid. In some embodiments, the acid is an inorganic acid. In some embodiments, the acid is aqueous sulfuric acid or hydrochloric acid. In some embodiments, the acid is an organic acid. In certain embodiments, the organic acid contains a carboxylic acid functionality. In some embodiments, the organic acid is propionic acid or acetic acid. In some embodiments, the acetic acid is aqueous acetic acid. In some embodiments, the acetic acid is glacial acetic acid. In some embodiments, the acid is added to the reaction mixture when the reaction is deemed to be over 50% complete, or over 60% complete, or over 70% complete, or over 80% complete, or over 90% complete, or over 95% complete, or over 98% complete, or over 99% complete.

In some embodiments, the reaction mixture is cooled prior to the addition of the acid, wherein said cooling refers to a drop in the temperature from the highest temperature reached during the reaction of the compound of formula (II) and the compound of formula (III). In some embodiments, the reaction mixture is cooled to less than 60° C., or less than 50° C., or less than 40° C. or about 30° C., or less than 20° C., or less than 10° C. or about 0° C. In some embodiments, the product is isolated by decantation of the reaction mixture. In some embodiments, the product is isolated by filtration of the reaction mixture. In some embodiments, the isolated product is washed with a solvent. In some embodiments, the product is washed with acetonitrile.

In some embodiments, the product of formula (I) is further purified by via recrystallization. In some embodiments, the recrystallization is performed by heating the compound of formula (I) in a solvent with activated carbon, and filtering through a medium to remove the activated carbon. In some embodiments, the recrystallization medium further comprises Celite. In some embodiments, the Celite is Celite 521. In some embodiments, the solvent for recrystallization comprises an organic solvent with a boiling point in excess of 70° C., or in excess of 80° C., or in excess of 90° C. In some embodiments, the solvent for recrystallization comprises a polar, non-protic solvent. In some embodiments the solvent comprises an ester, ketone, aromatic hydrocarbon or ether. In some embodiments, the aromatic hydro carbon is toluene. In some embodiments, the solvent is ethyl acetate, propyl acetate, or ethyl propionate. In some embodiments, the solvent is propyl acetate. In some embodiments, the recrsytallization mixture is filtered through Celite. In some embodiments, the filtrate is cooled and the desired compound of formula (I) is isolated. In some embodiments, a seed crystal of the compound of formula (I) is added to the recrystallization mixture in order to induce or accelerate crystallization. In some embodiments, the recrystallization, if deemed necessary, is performed in the absence of activated carbon or celite. In such a case, the hot filtrate typically does not need to be filtered, but rather can be directly cooled and then the product collected by filtration, decantation, or whatever other means deemed appropriate to the circumstance.

The term “alkyl” as used herein at all occurrences means both straight and branched chain radicals of 1 to 10 carbon atoms, unless the chain length is otherwise limited, including, but not limited to, methyl, ethyl, n-propyl, isopropyl, n-butyl, sec-butyl, isobutyl, tert-butyl, and the like.

The term “alkoxy” is used herein at all occurrences to mean a straight or branched chain radical of 1 to 8 carbon atoms, unless the chain length is limited thereto, bonded to an oxygen atom, including, but not limited to, methoxy, ethoxy, n-propoxy, isopropoxy, and the like.

The term “halogen” is used herein at all occurrences to mean chloro, fluoro, iodo and bromo.

The term “cycloalkyl” is used herein at all occurrences to mean cyclic radicals, preferably of 3 to 7 carbons, including but not limited to cyclopropyl, cyclopentyl, cyclohexyl, and the like.

The terms “aryl” or “heteroaryl” are used herein at all occurrences to mean substituted and unsubstituted aromatic ring(s) or ring systems which may include bi- or tri-cyclic systems and heteroaryl moieties, which may include, but are not limited to, heteroatoms selected from O, N, or S. Representative examples include, but are not limited to, phenyl, benzyl, naphthyl, pyridyl, quinolinyl, thiazinyl, and furanyl.

The term “optionally substituted” is used herein at all occurrences to mean that the moiety may be substituted or not, and if it is substituted, one or more hydrogen on each moiety is replaced with one or more substituents, each substituent being chosen independently from hydroxy, halogen, C₁₋₆ alkoxy or C₁₋₆ alkyl, as defined above.

A particularly preferred compound of formula (I) is (−)-(S)—N-(α-ethylbenzyl)-3-hydroxy-2-phenylquinoline-4-carboxamide. In some embodiments of this invention, the products of formula (I) are prepared and isolated as their free bases.

The compounds described herein may have asymmetric centers. Unless otherwise indicated, all chiral, diasteriomeric and racemic forms are included in the present invention. As is often the case, optimal therapeutic activity is provided only by one configuration of the two chiral centers. It is therefore desirable to produce this material in a form which is highly enriched in only one absolute configuration of the chiral centers. It is well known in the art how to prepare optically active compounds, such as by resolution of the racemic mixture, or by synthesis from optically active starting materials. In cases where a specific enantiomer is noted, for example S-1-phenylpropylamine, it is to be understood that this refers to a compound containing at least a predominance of the S-isomer. In some embodiments, the mixture will be at least 60% of the S-isomer, in some embodiments at least 70%, in some embodiments at least 80%, in some, in some embodiments at least 90%, in some embodiments at least 95%, in some embodiments at least 98% of the S-isomer.

Nomenclature is generally arrived at through the use of the ACD® naming plug-in to ISIS® desktop drawing software, or are generally accepted common names for compounds. The following examples are intended in no way to limit the scope of this invention.

### EXAMPLES OF PROCESSES OF THE INVENTION

### Preparation of (−)-(S)—N-(α-ethylbenzyl)-3-hydroxy-2-phenylquinoline-4-carboxamide

### Example 1

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (60.0 g, 0.226 mol, 1.00 eq), MeCN (240 mL, 4 vol) and triethylamine (41.0 mL, 0.294 mol, 1.30 eq) at room temperature with stirring. The mixture was stirred at room temperature until a solution was observed. Then 1,1′-carbonyldiimidazole (40.3 g, 0.249 mol, 1.10 eq) was charged in one portion. The mixture was heated to 25° C., and held at the same temperature for 5 h. Then S-1-phenylpropylamine (33.6 g, 0.249 mol, 1.10 eq) was charged in one portion at 25° C. The mixture was heated to 75° C., held at 75° C. for 5 h, cooled to room temperature, and stirred at room temperature overnight. At room temperature, glacial acetic acid (180 mL, 3.0 vol) was added in one portion, causing the solution temperature to rise to ˜38° C. The mixture was cooled slowly from 38° C. to 0° C. over ca. 4.5 h and the suspension was filtered through a filter paper under vacuum. The cake was washed with cold acetonitrile twice (2×120 mL, 2×2 vol) and de-ionized water once (120 mL, 2 vol) and dried under vacuum at 70° C. overnight to afford 73.4 g of (−)-(S)—N-(α-ethylbenzyl)-3-hydroxy-2-phenylquinoline-4-carboxamide as a light yellow to off-white solid, in 84.8% yield.

### Example 2

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (60.0 g, 0.226 mol, 1.00 eq), ACN (240 mL, 4 vol) and triethylamine (41.0 mL, 0.294 mol, 1.30 eq) at room temperature with stirring. The mixture was stirred at room temperature until a solution was observed. Then 1,1′-carbonyldiimidazole (40.3 g, 0.249 mol, 1.10 eq) was charged in one portion. The mixture was heated to 50° C., and held at the same temperature for 5 h. Then S-1-phenylpropylamine (33.6 g, 0.249 mol, 1.10 eq) was charged in one portion at 50° C. The mixture was held at 50° C. for 5 h, cooled to room temperature, and stirred at room temperature overnight. At room temperature, glacial acetic acid (180 mL, 3.0 vol) was added in one portion, causing the solution temperature to rise to ˜39° C. The mixture was cooled slowly from 39° C. to 0° C. over ca. 2 h and the suspension was filtered through a filter paper under vacuum. The cake was washed with cold acetonitrile twice (2×120 mL, 2×2 vol) and de-ionized water once (120 mL, 2 vol) and dried under vacuum at 70° C. overnight to afford 71.2 g of (−)-(S)—N-(α-ethylbenzyl)-3-hydroxy-2-phenylquinoline-4-carboxamide as a light yellow to off-white solid, in 82.3% yield.

### Example 3

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (60.0 g, 0.226 mol, 1.00 eq), ACN (240 mL, 4 vol) and triethylamine (41.0 mL, 0.294 mol, 1.30 eq) at room temperature with stirring. The mixture was stirred at room temperature until a solution was observed. Then 1,1′-carbonyldiimidazole (40.3 g, 0.249 mol, 1.10 eq) was charged in one portion. The mixture was heated to 37° C., and held at the same temperature for 5 h. Then S-1-phenylpropylamine (33.6 g, 0.249 mol, 1.10 eq) was charged at in one portion 37° C. The mixture was heated to 63° C., and held at 63° C. for 5 h, cooled to room temperature, and stirred at room temperature overnight. At room temperature, glacial acetic acid (180 mL, 3.0 vol) was added in one portion, causing the solution temperature to rise to ˜39° C. The mixture was cooled slowly from 39° C. to 0° C. over ca. 5 h and the suspension was filtered through a filter paper under vacuum. The cake was washed with cold acetonitrile twice (2×120 mL, 2×2 vol) and de-ionized water once (120 mL, 2 vol) and dried under vacuum at 70° C. overnight to afford 70.3 g of (−)-(S)—N-(α-ethylbenzyl)-3-hydroxy-2-phenylquinoline-4-carboxamide as a light yellow to off-white solid, in 81.2% yield.

### Example 4

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (60.0 g, 0.226 mol, 1.00 eq), ACN (240 mL, 4 vol) and triethylamine (41.0 mL, 0.294 mol, 1.30 eq) at room temperature with stirring. The mixture was stirred at room temperature until a solution was observed. Then 1,1′-carbonyldiimidazole (40.3 g, 0.249 mol, 1.10 eq) was charged in one portion. The mixture was heated to 25° C., and held at the same temperature for 5 h. Then S-1-phenylpropylamine (33.6 g, 0.249 mol, 1.10 eq) was charged in one portion at 25° C. The mixture was heated to 50° C., held at 50° C. for 5 h, cooled to room temperature, and stirred at room temperature overnight. At room temperature, glacial acetic acid (180 mL, 3.0 vol) was added in one portion, causing the solution temperature to rise to ˜39° C. The mixture was cooled slowly from 39° C. to 0° C. over ca. 5.5 h and the suspension was filtered through a filter paper under vacuum. The cake was washed with cold acetonitrile twice (2×120 mL, 2×2 vol) and de-ionized water once (120 mL, 2 vol) and dried under vacuum at 70° C. overnight to afford 76.4 g of (−)-(S)—N-(α-ethylbenzyl)-3-hydroxy-2-phenylquinoline-4-carboxamide as a light yellow to off-white solid, in 88.3% yield.

### Example 5

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (60.0 g, 0.226 mol, 1.00 eq), ACN (240 mL, 4 vol) and triethylamine (41.0 mL, 0.294 mol, 1.30 eq) at room temperature with stirring. The mixture was stirred at room temperature until a solution was observed. Then 1,1′-carbonyldiimidazole (40.3 g, 0.249 mol, 1.10 eq) was charged in one portion. The mixture was heated to 50° C., and held at the same temperature for 5 h. Then S-1-phenylpropylamine (33.6 g, 0.249 mol, 1.10 eq) was charged in one portion at 50° C. The mixture was heated to 75° C., held at 75° C. for 5 h, cooled to room temperature, and stirred at room temperature overnight. At room temperature, glacial acetic acid (180 mL, 3.0 vol) was added in one portion, causing the solution temperature to rise to ˜38° C. The mixture was cooled slowly from 38° C. to 0° C. over ca. 5 h and the suspension was filtered through a filter paper under vacuum. The cake was washed with cold acetonitrile twice (2×120 mL, 2×2 vol) and de-ionized water once (120 mL, 2 vol) and dried under vacuum at 70° C. overnight to afford 69.1 g of (−)-(S)—N-(α-ethylbenzyl)-3-hydroxy-2-phenylquinoline-4-carboxamide as a light yellow to off-white solid, in 79.9% yield.

### Example 6

To a reaction vessel under nitrogen atmosphere were charged 3-hydroxy-2-phenyl-4-quinolinecarboxylic acid (60.0 kg, 1 equivalent) and acetonitrile (2400 L, 4 volumes). Triethylamine (29.8 kg, 1.3 equivalents) was added at ambient temperature. The reaction mixture was stirred for ˜20 min at 20-35° C. to a cloudy brown solution. 1,1′-carbonyldiimidazole (CDI) (40.3 kg, 1.1 equivalents) was charged in one portion. The reaction mixture was then heated to 40-50° C. and held at 40-50° C. for 2-4 hours. Reaction was monitored by HPLC analysis of In-Process-Monitoring (IPM) samples. IPM sample was quenched with HPLC grade methanol immediately. When IPM results indicate the ratio of methyl 3-hydroxy-2-phenyl-4-quinolinecarboxylate and 3-hydroxy-2-phenyl-4-quinolinecarboxylic acid was >98:1, the first reaction was complete. S-1-phenylpropylamine (33.6 kg, 1.1 equivalents) was added in one portion at 40-50° C. The reaction was heated to 70-75° C. under nitrogen and held for 2-3 hours at 70-75° C. The reaction was deemed complete when IPM results indicate the ratio of (−)-(S)—N-(α-ethylbenzyl)-3-hydroxy-2-phenylquinoline-4-carboxamide to methyl 3-hydroxy-2-phenyl-4-quinolinecarboxylate was greater than 99.5:1. The reaction mixture was cooled to 20-25° C. and filtered through in-line filter. Glacial acetic acid (1800 L, 3 volumes) was added to the above reaction mixture, while keeping the process temperature at 40-55° C. The reaction mixture was stirred for 1 hour at 35-45° C. and then cooled slowly to approx. 0° C. at 1° C. per minute. The resulting slurry was stirred for 2 hrs at approx. 0° C. The crude product was isolated by filtration in centrifuge. The wet cake was washed with cold acetonitrile (approx. 0° C., 120 L, 2 volumes) twice and deionized water (120 L, 2 volumes). The crude product was dried in vacuum oven at 60-70° C. under vacuum. Yield: 69.2 kg, 80.1%; and 73.9 kg, 85.5%

### Example 7

To a 1000 mL 3-necked round bottom flask equipped with air-driven mechanical stirrer, thermometer, reflux condenser, addition funnel and nitrogen inlet/outlet, were added 3-hydroxy-2-phenyl-4-quinolinecarboxylic acid (32.0 g, 0.121 mol, 1.00 eq), ACN (128 mL, 4 vol) and triethylamine (21.8 mL, 0.156 mol, 1.30 eq) at room temperature with stirring. The mixture was stirred at room temperature until a solution was observed. Then 1,1′-carbonyldiimidazole (21.5 g, 0.133 mol, 1.10 eq) was charged in one portion. The mixture was heated to 50° C., and held at the same temperature for 2 h. Then S-1-phenylpropylamine (17.9 g, 0.133 mol, 1.10 eq) was charged in one portion at 50° C. The mixture was heated to 75° C., held at 75° C. for 4 h, cooled to room temperature, and stirred at room temperature overnight. The reaction mixture was filtered through 1 micron paper in a Buchner funnel. The filtrate was heated to 30° C. Glacial acetic acid (prefiltered through 1 micron paper, 96 mL, 3.0 vol) was added in one portion, causing the solution temperature to rise to ˜47° C. The mixture was cooled slowly from 47° C. to 0° C. over ca. 3 h and the suspension was filtered through a filter paper under vacuum. The cake was washed with cold acetonitrile twice (2×64 mL, 2×2 vol) and de-ionized water once (64 mL, 2 vol) and dried under vacuum at 60° C. overnight to afford 37 g of (−)-(S)—N-(α-ethylbenzyl)-3-hydroxy-2-phenylquinoline-4-carboxamide as a light yellow to off-white solid, in 82.3% yield. m.p. 122-125° C. 1H-NMR (400 MHz, DMSO-d6): 9.81 (s, 1H), 9.15 (d, 1H), 7.96-8.02 (m, 3H), 7.44-7.63 (m, 8H), 7.36-7.42 (m, 2H), 7.26-7.32 (m, 1H), 5.01-5.08 (q, 1H), 1.72-1.84 (m, 3H), 0.93-0.98 (t, 3H).

### Example 8

### Recrystallization of (−)-(S)—N-(α-ethylbenzyl)-3-hydroxy-2-phenylquinoline-4-carboxamide

To a reaction vessel were charged crude (−)-(S)—N-(α-ethylbenzyl)-3-hydroxy-2-phenylquinoline-4-carboxamide (120 kg, 1 equivalent), activated carbon (6 kg, 5% w/w, 60-100 mesh), and n-propyl acetate (960 L, 8 volumes). The contents were heated up to gentle reflux (95-100° C.) and held for about 30 min. The suspension was then cooled to around 70-80° C. and filtered through a Celite pad and in-line filter. The reactor, filters and lines were washed with hot n-propyl acetate (120 L, 1 volume, 70-80° C.). The filtrate was concentrated down to approximately 4 volumes by atmospheric distillation (102-107° C.). The solution was cooled (1.0-1.5° C./min) to 74-78° C. and seeded with seed crystals of (−)-(S)—N-(α-ethylbenzyl)-3-hydroxy-2-phenylquinoline-4-carboxamide (840 g, 0.7% w/w) and held for 60 minutes at 75° C. The contents were cooled to −2 to 2° C. over 75-150 minutes (0.5 to 1.0° C./min.) and held for at least 30 min. The resulting slurry was filtered in the filter drier under approximately 0.5-1.0 bar G pressure. The cake was washed twice with heptane (240 L, 2 volumes) via the reactor. The solid product was dried in the filter drier at ambient temperature under 0.5-1.0 bar G pressure. Yield 105.8 kg, 88.0% of white crystalline solid.

### Example 9

To a 250 mL 3-necked round bottom flask equipped with air-driven mechanical stirrer and thermometer was added 30 g of (−)-(S)—N-(α-ethylbenzyl)-3-hydroxy-2-phenylquinoline-4-carboxamide. N-Propyl acetate (prefiltered through 1 micron paper, 120 mL, 4 vol) was added and the resulting slurry was heated to 100° C. After a clear solution was obtained, the solution was cooled from 100° C. to 0° C. over 2-3 h. The crystals were filtered via vacuum and washed with heptane (60 mL, 2 vol) then dried under vacuum at 60° C. overnight to afford 25 g of (−)-(S)—N-(α-ethylbenzyl)-3-hydroxy-2-phenylquinoline-4-carboxamide, in 83% yield.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A method of preparation of a compound of formula (I) 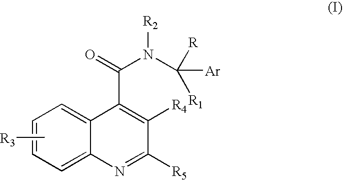
or a pharmaceutically acceptable salt form thereof, wherein:
Ar is an optionally substituted phenyl group, or a naphthyl or C₅₋₇ cycloalkdienyl group, or an optionally substituted single or fused ring heterocyclic group, having aromatic character, containing from 5 to 12 ring atoms and comprising up to four hetero-atoms selected from S, O, N;
R is linear or branched C₁₋₈ alkyl, C₃₋₇ cycloalkyl, C₄₋₇ cycloalkylalkyl, an optionally substituted phenyl group or a phenyl C₁₋₆ alkyl group, an optionally substituted five-membered heteroaromatic ring comprising up to four heteroatom selected from O and N, hydroxy C₁₋₆ alkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl; or is a group —(CH₂)ₚ— when cyclized onto Ar, where p is 2 or 3;
R₁ and R₂, which may be the same or different, are independently hydrogen or C₁₋₆ linear or branched alkyl, or together form a —(CH₂)ₙ— group in which n represents 3, 4, or 5; or R₁ together with R forms a group —(CH₂)_{q}—, in which q is 2, 3, 4 or 5;
R₃ may be hydrogen, C₁₋₆ linear or branched alkyl, C₁₋₆ alkenyl, aryl, halogen, nitro, cyano, carboxy, carboxamido, sulphonamido, trifluoromethyl, amino, mono- and di-C₁₋₆ alkylamino, —O(CH₂)ᵣ—NT₂, in which r is 2, 3, or 4 and T is C₁₋₆ alkyl or it forms a heterocyclic group 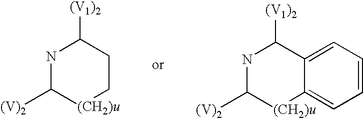
in which V and V₁ are hydrogen and u is 0, 1 or 2;
R₄ is hydroxyl;
R₅ is branched or linear C₁₋₆ alkyl, C₃₋₇ cycloalkyl, C₄₋₇ cycloalkylalkyl, optionally substituted aryl, wherein the optional substituent is one of hydroxy, halogen, C₁₋₆ alkoxy or C₁₋₆ alkyl, or an optionally substituted single or fused ring heterocyclic group, having aromatic character, containing from 5 to 12 ring atoms and comprising up to four hetero-atoms selected from S, O, N, comprising:
a) contacting a compound of formula (IV) with 1,1′-carbonyldiimidazole 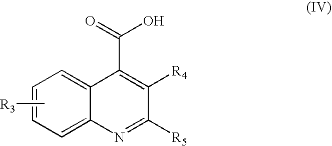
in the presence of optional base and optional solvent; and
b) contacting the product of step a) with a compound of formula (III)

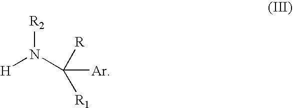

2. The method of claim 1, wherein the compound of formula (I) is (−)-(S)—N-(α-ethylbenzyl)-3-hydroxy-2-phenylquinoline-4-carboxamide or a salt or solvate thereof, the compound of formula (IV) is 3-hydroxy-2-phenyl-4-quinolinecarboxylic acid or a salt or solvate thereof, and the compound of formula (III) is S-1-phenylpropylamine or a salt or solvate thereof.

3. The method of claim 2, wherein a solvent is used and said solvent comprises an ether, aromatic hydrocarbon, alkylnitrile, or ester.

4. The method of claim 3, wherein said solvent comprises tetrahydrofuran, acetonitrile, toluene, or n-propylacetate.

5. The method of claim 4, wherein said solvent comprises acetonitrile.

6. The method of claim 3, wherein step a) is performed in the presence of a base.

7. The method of claim 6, wherein said base is an amine.

8. The method of claim 7, wherein said amine is selected from the group consisting of 2,6-lutidine, 2,4,6-collidine, 2,3-lutidine, pyrazine, pyridine, N-methylpyrrole, DBN (1,5-Diazabicyclo[4.3.0]non-5-ene), DBU (diazabicyclo[5.4.0]undec-7-ene), imidazole, DMAP (4-dimethylaminopyridine), triethylamine, N,N-dimethylethylamine, N-methylmorpholine, diisopropylethylamine, diisopropylamine, 2,6-dimethylpiperidine, tributylamine, and dicyclohexylamine, and combinations thereof.

9. The method of claim 8, wherein said amine is selected from the group consisting of imidazole, N-methylmorpholine, or triethylamine, or a combination thereof.

10. The method of claim 9, wherein said amine is triethylamine.

11. The method of claim 10, wherein said triethylamine is used in greater than 1 equivalent.

12. The method of claim 9, wherein an acid is added to the reaction mixture.

13. The method of claim 12, wherein said acid is glacial acetic acid.

14. The method of claim 5, wherein for step a) the reaction mixture comprising 3-hydroxy-2-phenyl-4-quinolinecarboxylic acid, 1,1′-carbonyldiimidazole, triethylamine, and acetonitrile is heated to about 40 to 50° C.; and wherein said triethylamine is present in more than 1 equivalent.

15. The method of claim 14, wherein for step b), S-1-phenylpropylamine is added to the reaction mixture from step a) and heated to about 70 to 75° C.

16. The method of claim 15, wherein the reaction mixture is treated with glacial acetic acid.

17. A compound of formula (II) 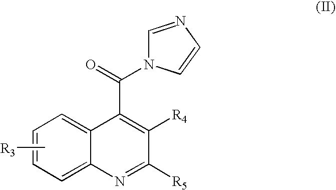
wherein R₃, R₄ and R₅ are as defined in claim 1.

18. A compound of formula (II) of claim 17, wherein the compound is 4-(1H-imidazol-1-ylcarbonyl)-2-phenyl-3-quinolinol.

19. A mixture containing a compound of claim 18 and 3-hydroxy-2-phenyl-4-quinolinecarboxylic acid, wherein the molar ratio of the compound of claim 18 and 3-hydroxy-2-phenyl-4-quinolinecarboxylic acid is greater than ½.

20. A method of preparing a compound of claim 17, wherein said method comprises contacting a compound of formula (IV) 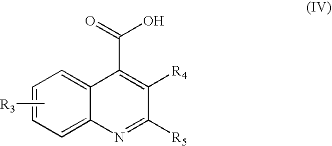
with 1,1′ carbonyldiimidazole.

21. The method of claim 20, wherein the compound of formula (IV) is 3-hydroxy-2-phenyl-4-quinolinecarboxylic acid.

22. A method of preparing a compound of formula (I) of claim 1, 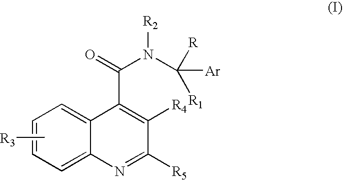
comprising the contacting of a compound of formula (II) of claim 17 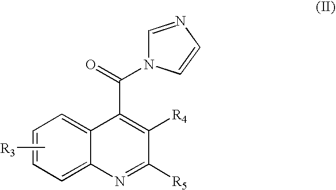
with a compound of formula (III) of claim 1, 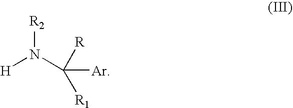
wherein Ar, R, R₁, R₂, R₃, R₄, and R₅ are as defined in claim 1.

23. A method of purification of (−)-(S)—N-(α-ethylbenzyl)-3-hydroxy-2-phenylquinoline-4-carboxamide comprising heating a mixture of (−)-(S)—N-(α-ethylbenzyl)-3-hydroxy-2-phenylquinoline-4-carboxamide in a solvent system comprising an ester followed by cooling and isolating the product.

24. The method of claim 23, wherein said ester is n-propyl acetate.

25. The method of claim 24, wherein said mixture further contains activated carbon.
